SEQUENCE LISTING (34 sequences)

SEQ ID NO: 1 - RNA, 25 nt, Artificial Sequence
  Description of Artificial Sequence Synthetic oligonucleotide
ggauccacgagcccaagggcuacca

SEQ ID NO: 2 - RNA, 25 nt, Artificial Sequence
  Description of Artificial Sequence Synthetic oligonucleotide
ugguagcccuugggcucguggaucc

SEQ ID NO: 3 - RNA, 25 nt, Artificial Sequence
  Description of Artificial Sequence Synthetic oligonucleotide
cccaagggcuaccaugccaacuucu

SEQ ID NO: 4 - RNA, 25 nt, Artificial Sequence
  Description of Artificial Sequence Synthetic oligonucleotide
agaaguuggcaugguagcccuuggg

SEQ ID NO: 5 - RNA, 25 nt, Artificial Sequence
  Description of Artificial Sequence Synthetic oligonucleotide
gagcccaagggcuaccaugccaacu

SEQ ID NO: 6 - RNA, 25 nt, Artificial Sequence
  Description of Artificial Sequence Synthetic oligonucleotide
aguuggcaugguagcccuugggcuc

SEQ ID NO: 7 - RNA, 25 nt, Artificial Sequence
  Description of Artificial Sequence Synthetic oligonucleotide
gauccacgagcccaagggcuaccau

SEQ ID NO: 8 - RNA, 25 nt, Artificial Sequence
  Description of Artificial Sequence Synthetic oligonucleotide
augguagcccuugggcucguggauc

SEQ ID NO: 9 - RNA, 25 nt, Artificial Sequence
  Description of Artificial Sequence Synthetic oligonucleotide
cacgagcccaagggcuaccaugcca

SEQ ID NO: 10 - RNA, 25 nt, Artificial Sequence
  Description of Artificial Sequence Synthetic oligonucleotide
uggcaugguagcccuugggcucgug

SEQ ID NO: 11 - RNA, 25 nt, Artificial Sequence
  Description of Artificial Sequence Synthetic oligonucleotide
gaggucacccgcgugcuaauggugg

SEQ ID NO: 12 - RNA, 25 nt, Artificial Sequence
  Description of Artificial Sequence Synthetic oligonucleotide
ccaccauuagcacgcgggugaccuc

SEQ ID NO: 13 - RNA, 25 nt, Artificial Sequence
  Description of Artificial Sequence Synthetic oligonucleotide
guacaacagcacccgcgaccgggug

SEQ ID NO: 14 - RNA, 25 nt, Artificial Sequence
  Description of Artificial Sequence Synthetic oligonucleotide
cacccggucgcgggugcuguuguac

SEQ ID NO: 15 - RNA, 25 nt, Artificial Sequence
  Description of Artificial Sequence Synthetic oligonucleotide
guggauccacgagcccaagggcuac

SEQ ID NO: 16 - RNA, 25 nt, Artificial Sequence
  Description of Artificial Sequence Synthetic oligonucleotide
guagcccuugggcucguggauccac

SEQ ID NO: 17 - protein, 17 aa, Artificial Sequence
  Description of Artificial Sequence Synthetic peptide
KHHHKHHHKHHHKHHHK

SEQ ID NO: 18 - protein, 18 aa, Artificial Sequence
  Description of Artificial Sequence Synthetic peptide
KHHHKHHHKHHHHKHHHK

SEQ ID NO: 19 - RNA, 25 nt, Artificial Sequence
  Description of Artificial Sequence Synthetic oligonucleotide
ggucuggugccuggucugaugaugu

SEQ ID NO: 20 - RNA, 25 nt, Artificial Sequence
  Description of Artificial Sequence Synthetic oligonucleotide
acaucaucagaccaggcaccagacc

SEQ ID NO: 21 - RNA, 25 nt, Artificial Sequence
  Description of Artificial Sequence Synthetic oligonucleotide
gagcaccauucuccuugaaaggacu

SEQ ID NO: 22 - RNA, 25 nt, Artificial Sequence
  Description of Artificial Sequence Synthetic oligonucleotide
aguccuuucaaggagaauggugcuc

SEQ ID NO: 23 - RNA, 25 nt, Artificial Sequence
  Description of Artificial Sequence Synthetic oligonucleotide
ccucaauucagucucucaucugcaa

SEQ ID NO: 24 - RNA, 25 nt, Artificial Sequence
  Description of Artificial Sequence Synthetic oligonucleotide
uugcagaugagagacugaauugagg

SEQ ID NO: 25 - RNA, 25 nt, Artificial Sequence
  Description of Artificial Sequence Synthetic oligonucleotide
gauguuugcauucuuugcccagcac

SEQ ID NO: 26 - RNA, 25 nt, Artificial Sequence
  Description of Artificial Sequence Synthetic oligonucleotide
gugcugggcaaagaaugcaaacauc

SEQ ID NO: 27 - RNA, 25 nt, Artificial Sequence
  Description of Artificial Sequence Synthetic oligonucleotide
gucuuuggucuggugccuggucuga

SEQ ID NO: 28 - RNA, 25 nt, Artificial Sequence
  Description of Artificial Sequence Synthetic oligonucleotide
ucagaccaggcaccagaccaaagac

SEQ ID NO: 29 - RNA, 25 nt, Artificial Sequence
  Description of Artificial Sequence Synthetic oligonucleotide
gugccuggucugaugauguaugcca

SEQ ID NO: 30 - RNA, 25 nt, Artificial Sequence
  Description of Artificial Sequence Synthetic oligonucleotide
uggcauacaucaucagaccaggcac

SEQ ID NO: 31 - RNA, 25 nt, Artificial Sequence
  Description of Artificial Sequence Synthetic oligonucleotide
caccauucuccuugaaaggacuuau

SEQ ID NO: 32 - RNA, 25 nt, Artificial Sequence
  Description of Artificial Sequence Synthetic oligonucleotide
auaaguccuuucaaggagaauggug

SEQ ID NO: 33 - RNA, 25 nt, Artificial Sequence
  Description of Artificial Sequence Synthetic oligonucleotide
caauucagucucucaucugcaauaa

SEQ ID NO: 34 - RNA, 25 nt, Artificial Sequence
  Description of Artificial Sequence Synthetic oligonucleotide
uuauugcagaugagagacugaauug

(19) United States
(12) Patent Application Publication
(10) Pub. No.: US 2021/0324384 A1
(43) Pub. Date: Oct. 21, 2021

(54) SILENCING TGF-BETA 1 and COX2 USING siRNAs DELIVERED in a POLYPEPTIDE NANOPARTICLE ALONE and in COMBINATION with IMMUNE CHECKPOINT INHIBITORS to TREAT CANCER

(71) Applicant: Sirnaomics, Inc., Gaithersburg, MD (US)

(72) Inventors: David M. EVANS, Gaithersburg, MD (US); Patrick Y. LU, Gaithersburg, MD (US)

(21) Appl. No.: 17/361,109

(22) Filed: Jun. 28, 2021

Related U.S. Application Data
(63) Continuation of application No. PCT/US2019/068499, filed on Dec. 24, 2019.
(60) Provisional application No. 62/785,647, filed on Dec. 27, 2018.

(51) Int. Cl. C12N 15/113; A61K 9/51; A61K 31/713; A61K 39/395; C07K 16/28; A61P 35/00

(52) U.S. Cl. CPC C12N 15/1136; C12N 15/1137; A61K 9/5169; A61K 31/713; C12N 2310/14; C07K 16/2827; A61P 35/00; C12N 2320/31; A61K 39/3955

(57) ABSTRACT

The present invention provides certain pharmaceutical molecules and compositions and methods of using them to treat cancer. The molecules are small interfering RNA (siRNA) molecules that inhibit TGF-beta 1 and Cox2 in humans and other mammals, which are used alone or in combination with immune checkpoint inhibitors, to treat cancer.

### CROSS-REFERENCE TO RELATED PATENT APPLICATIONS

This application is a continuation of International Application No. PCT/US2019/068499, filed on Dec. 24, 2019, which claims the benefit of and priority to U.S. Provisional Patent Application No. 62/785,647, filed Dec. 27, 2018, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Feb. 14, 2020, is named 4690_0016 i_SL.txt and is 8,518 bytes in size.

### FIELD OF THE INVENTION

The invention relates to certain pharmaceutical molecules and compositions and their use to treat cancer and, in particular, to the use of small interfering RNA (siRNA) molecules that inhibit TGF-beta 1 and Cox2, alone or in combination with immune checkpoint inhibitors, to treat cancer.

### BACKGROUND

Recent work has identified immune checkpoint inhibitors as important targets in the fight against cancer. Receptors such as PD-1 (on the surface of T-cells) can interact with ligands (e.g PD-L1) on the surface of tumor cells, and this binding between these molecules results in a signal to the T-cell that the tumor should not be destroyed. Consequently, this signaling mechanism has been thoroughly studied to try and identify ways to block it and, therefore, promote the immune recognition of tumors, with the resulting increase in tumor killing that this produces.

There are many checkpoints that have been discovered, including CTLA4, Lag3, Tim3, PD-1 and PD-L1. Antibodies against PD1 or against PD-L1, for example, have been demonstrated to block the interaction between the PD-1 receptor and the PD-L1 ligand, and this inhibits the “do not eat me” signal from the tumor cell to the T-cell, which otherwise prevents the T-cell from enacting its normal response to tumors and other foreign cells by releasing enzymes that kill the cells.

With the migration of these antibodies (pembrolizumab, Keytruda, etc.) to the clinic, it was found that treating patients with these agents could promote a very strong immune response in about 30% of patients that resulted in a long-lasting cure of those patients. However, it was not clearly understood why this response was seen in only 30% of the patients treated, and so research has focused extensively on other pathways and signaling mechanisms that may be at play to inhibit the immune reaction and the ability of T-cells to kill tumor cells.

RNA interference (RNAi) is a sequence-specific RNA degradation process that provides a relatively easy and direct way to knockdown, or silence, theoretically any gene containing the homologous sequence. In naturally occurring RNAi, a double-stranded RNA (dsRNA) is cleaved by an RNase III/helicase protein, Dicer, into small interfering RNA (siRNA) molecules, dsRNA of 19-27 nucleotides (nt) with 2-nt overhangs at the 3′ ends. Afterwards, the siRNAs are incorporated into a multicomponent-ribonuclease called RNA-induced-silencing-complex (RISC). One strand of siRNA remains associated with RISC to guide the complex towards a cognate RNA that has a sequence complementary to the guider ss-siRNA in RISC. This siRNA-directed endonuclease digests the RNA, resulting in truncation and inactivation of the targeted RNA. Recent studies have revealed the utility of chemically synthesized 21-27-nt siRNAs that exhibit RNAi effects in mammalian cells and have demonstrated that the thermodynamic stability of siRNA hybridization (at terminals or in the middle) plays a central role in determining the molecule's function. More detailed characteristics of RISC, siRNA molecules, and RNAi have been described in the scientific literature.

The utility of RNAi in down-regulation of mammalian cell gene expression has been shown successfully in the laboratory by utilizing either chemically synthesized siRNAs or endogenously expressed siRNA. The endogenous siRNA is first expressed as small hairpin RNAs (shRNAs) by an expression vector (plasmid or virus vector), and then processed by Dicer to become functional siRNAs.

In order to have activity and be able to silence the target genes, siRNAs can be delivered to the cells where the silencing must occur by transfection of these cells. One way to get siRNA delivery to these cells is to use a nanoparticle that can carry the siRNAs and allow uptake of the siRNAs across the external cell membrane, gaining access to the cytoplasm. Release of siRNAs into the cytoplasm allows these moieties to interact with the RISC complex, where the antisense strand is separated from the sense strand, the sense strand is degraded, and the antisense strand is used by the RISC complex to surveil the mRNAs within the cell for a sequence with complementarity to the antisense sequence. This allows the hybridization of the two sequences, and the cleavage of the mRNA then occurs through the action of the enzyme Dicer.

mRNA provides the template responsible for translation of the mRNA sequence into a viable protein needed by the cell. Cleavage of the mRNA reduces the ability of the cell to produce the peptide or protein encoded by the mRNA. Silencing of the genes by siRNA can exhibit a prolonged effect and is dependent on the turnover and balance between the rate of synthesis, the quantity of mRNA, and the rate of degradation of the mRNA and/or the protein itself. SiRNAs have been shown to have a potent silencing effect on the gene targeted, and this can further result in a prolonged decrease (days to weeks) of the protein product.

Elevated TGF-beta 1 levels have been implicated in inhibiting the penetration of T-cells into the regions in proximity to tumors [1-3].

For example, in tumors from patients with colon cancer [1,2] or metastatic urothelial cancer who were treated with an anti-PD-L1 antibody (atezolizumab), a lack of response was associated with transforming growth factor β (TGFβ) signaling in fibroblasts. Primarily this was observed in tumors which showed exclusion of CD8+ T-cells from the tumor parenchyma and accumulation of these T-cells in the fibroblast- and collagen-rich peritumoural stroma, a common phenotype among patients with metastatic urothelial cancer. Co-administration of TGFβ-blocking antibody with anti-PD-L1 antibodies reduced TGFβ signaling in stromal cells, and this, in turn, allowed T-cell penetration into the center of the tumors—provoking anti-tumor immunity and tumor regression in this model [3].

While [1] and [2] focused on colon cancer and [3] shows effects in urothelial cancer, nobody had tried to look at the synergy between TGFβ1 AND PDL1 inhibition on an immune response when both targets were inhibited at the same time. We used a Polypeptide Nano Particle (PNP) to deliver siRNAs targeting TGFβ1 and Cox2 to the liver to see if this effect would improve efficacy of PDL1 antibodies in this disease. Delivery to the endothelial cells as well as the normal hepatocytes suggested we could silence the two genes within the vicinity of the tumor.

### BRIEF DESCRIPTION OF THE FIGURES

FIG. 1. Localization of IV administered STP707 within the cells of the liver. AF647 fluorescent-labeled siRNA was formulated with HKP to form a nanoparticle. The nanoparticles were administered in mice through IV injection (tail vein administration). At the time points noted in the figure, the animals were euthanized, livers excised and dissected and dissociated cells were subjected to flow cytometry with labeled antibodies able to discriminate the different cell types shown. Cell types are shown from left to right under each time point Kupffer cells (KC), Dendritic Cells (DC), Liver Sinusoidal Endothelial cells (LSEC), Hepatocytes, Ly6c High (inflammatory monocytes), Ly6c Low, PolyMorphoNuclear leucocyte cells (PMN), Lymphocytes and Stellate Cells. This figure shows that hepatocytes, Kupffer cells and LSEC cells initially take up STP707 by 1 h. The stellate cell population within the liver is only 1.4% of the cell population. While the flow cytometry showed some evidence of uptake, the signal was too low, so we validated uptake in primary human stellate cells and showed excellent uptake and gene silencing within these cells.

FIG. 2. Using luciferin substrate administered to anesthetized animals, the amount of tumor was assessed by measuring the light efflux from each animal (measured using a digital imaging system). Animals were assigned to a cohort based on the normalization of tumor across all test groups. This figure shows the initial values produced at assignment to the groups—showing uniformity of tumor size between cohorts prior to commencing treatments.

FIG. 3. Body weights of the animals were monitored after each treatment and the body weights averaged across all animals in the cohort. The data was plotted as % of initial bodyweight prior to dosing.

Sorafenib alone (red squares) induced a slight change in body weights. However, all other treatment schemes (STP707 alone or +anti-PDL1) were well tolerated and no significant body weight loss was observed in the treatment arms.

FIG. 4. Tumor Associated BioLuminescence (TABL) measurements were made by administration of the substrate for luciferase (luciferin) to anesthetized animals and then imaging the light generated with an IVIS live animal imaging system. Animals were administered treatments throughout the dosing phase (Grey highlighted region) and were otherwise monitored without treatment at the other times.

Control (vehicle) treated animals showed a rapid growth of tumors as determined by a larger light signal. Sorafenib and anti-PDL1 mAb treatments appeared to show a static effect on tumor growth over the dosing phase. STP707 alone (40 ug per injection or ^{˜}2 mgs/kg) or with the Anti-PDL1 mAb showed a dramatic reduction in tumor cells after 5-6 doses. Tumors were not visible in these treatment arms.

FIG. 5. Control (vehicle treated) animals showed a dramatic effect of uncontrolled tumor growth on viability of the animals. 50% of the untreated animals died or were euthanized as a result of increased tumor burden during the dosing phase of the experiment. 1 animal was euthanized after 37 days on Sorafenib. No animals died on any of the other treatment arms.

FIG. 6. Data shows that control samples (PNP loaded with Non-Silencing (NS) siRNA) showed a dramatic increase in tumor cell growth as measured by the Flux reading from outside the animal using an IVIS imaging system. PDL1 antibody showed a weak inhibitory effect on tumor growth. STP707 alone (1 mg/Kg) showed an even greater inhibition of tumor growth than the PDL1 Ab and, in the presence of the anti-PDL1 mAb, STP707 abolished the tumor completely after 6 doses—suggesting some additivity of effect with the antibody.

FIG. 7. STP707 was administered at 3 doses at 1 mg/kg in animals with a syngeneic orthotopic HCC tumor and then livers were excised, sectioned and stained (H&E) to show tumor location and size. Note the dramatic reduction in tumor size when STP707 was administered for this short period of time. In the regions shown by the white boxes, the amount of CD4+ and CD8+ T-cells were quantitated by staining and counting the stained spots. These regions are expanded on the right of each figure and it can clearly be seen that STP707 treatment produced a dramatic increase in the number of CD4+ and CD8+ T-cells present within the liver-tumor margin—suggesting that STP707 treatment allows greater T-cell penetration into the tumor.

FIG. 8. Using images similar to those shown in FIG. 7, T-cells were quantitated in various segments measured away from the tumor margin—either in towards the tumor, or out towards the liver. Within each segment (50 um thick), the number of T-cells were quantitated and plotted on the graph shown. STP707 treatment together with the anti-PDL1 Ab showed a 2-fold increase in CD8+ T-cells at 500 um depth into the tumor compared with vehicle treatment alone. It also shows an increase in CD8+ T-cells within the liver close to the tumor—suggesting possible recruitment of T-cells induced by the lowering of the TGF-beta “wall” surrounding the tumor.

### DESCRIPTION OF THE INVENTION

The invention relates to the use of small interfering RNA (siRNA) molecules that inhibit TGF-beta 1 and Cox2 in a subject, alone or in combination with immune checkpoint inhibitors, to treat cancer in the subject. As used herein, the term “subject” refers to any mammal, including humans. The subject may be a laboratory animal, such as a rodent, ferret, or non-human primate. Preferably, the subject is a human.

In one embodiment, the invention is directed to a method of killing cancer cells in a subject by administering to the subject a therapeutically effective amount of an anti-TGF-beta 1 siRNA. In one aspect of this embodiment, anti-TGF-beta 1 siRNA is administered intravenously to the subject. In another aspect of this embodiment, anti-TGF-beta 1 siRNA is administered into a tumor in the subject. In still another aspect of this embodiment, anti-TGF-beta 1 siRNA is administered in proximity to the tumor or administered systemically in a vehicle that allows delivery to the tumor.

In another embodiment, the invention is directed to a method of treating a cancer in a subject by administering to the subject a therapeutically effective amount of an anti-TGF-beta 1 siRNA. In one aspect of this embodiment, anti-TGF-beta 1 siRNA is administered intravenously to the subject. In another aspect of this embodiment, anti-TGF-beta 1 siRNA is administered into a tumor in the subject. In still another aspect of this embodiment, anti-TGF-beta 1 siRNA is administered in proximity to the tumor or administered systemically in a vehicle that allows delivery to the tumor.

The cancer (and the cancer cells) are any cancer that afflicts a subject. Such cancers include liver, colon, pancreatic, lung, and bladder cancer. The liver cancer can be a primary liver cancer or a cancer that has metastasized to the liver from another tissue. Primary liver cancers include hepatocellular carcinoma and hepatoblastoma. Metastasized cancers include colon and pancreatic cancer.

The anti-TGF-beta 1 siRNA molecules include the sequences identified in Table 1.

TABLE 1
Anti-TGFbeta 1 siRNA Sequences

| | |
|---|---|
| hmTF-25-1: | sense 5′-r(GGAUCCACGAGCCCAAGGGCUACCA)-3′ (SEQ ID NO: 1) |
| | antisense 5′-r(UGGUAGCCCUUGGGCUCGUGGAUCC)-3 (SEQ ID NO: 2) |
| hmTF-25-2: | sense 5′-r(CCCAAGGGCUACCAUGCCAACUUCU)-3′ (SEQ ID NO: 3) |
| | antisense 5′-r(AGAAGUUGGCAUGGUAGCCCUUGGG)-3′ (SEQ ID NO: 4) |
| hmTF-25-3: | sense 5′-r(GAGCCCAAGGGCUACCAUGCCAACU)-3′ (SEQ ID NO: 5) |
| | antisense 5′-r(AGUUGGCAUGGUAGCCCUUGGGCUC)-3′ (SEQ ID NO: 6) |
| hmTF25-4: | sense, 5'-r(GAUCCACGAGCCCAAGGGCUACCAU)-3′ (SEQ ID NO: 7) |
| | antisense, 5′-r(AUGGUAGCCCUUGGGCUCGUGGAUC)-3′ (SEQ ID NO: 8) |
| hmTF25-5: | sense, 5′-r(CACGAGCCCAAGGGCUACCAUGCCA)-3′ (SEQ ID NO: 9) |
| | antisense, 5′-r(UGGCAUGGUAGCCCUUGGGCUCGUG)-3′ (SEQ ID NO: 10) |
| hmTF25-6: | sense, 5′-r(GAGGUCACCCGCGUGCUAAUGGUGG)-3′ (SEQ ID NO: 11) |
| | antisense, 5′-r(CCACCAUUAGCACGCGGGUGACCUC)-3′ (SEQ ID NO: 12) |
| hmTF25-7: | sense, 5′-r(GUACAACAGCACCCGCGACCGGGUG)-3′ (SEQ ID NO: 13) |
| | antisense, 5′-r(CACCCGGUCGCGGGUGCUGUUGUAC)-3′ (SEQ ID NO: 14) |
| hmTF25-8: | sense, 5′-r(GUGGAUCCACGAGCCCAAGGGCUAC)-3′ (SEQ ID NO: 15) |
| | antisense, 5′-r(GUAGCCCUUGGGCUCGUGGAUCCAC)-3′ (SEQ ID NO: 16) |

In one aspect, the anti-TGF-beta 1 siRNA comprises the following sequences: sense: 5′-cccaagggcuaccaugccaacuucu-3′ (SEQ ID NO: 3); antisense: 5′-agaaguuggcaugguagcccuuggg-3′ (SEQ ID NO: 4).

The anti-TGF-beta 1 siRNA is administered to the subject in a pharmaceutically acceptable carrier. Such carriers include branched histidine-lysine polymers. In one embodiment of such polymers, the polymer has the formula (R)K(R)-K(R)-(R)K(X), where R=KHHHKHHHKHHHKHHHK (SEQ ID NO: 17) or R=KHHHKHHHKHHHHKHHHK (SEQ ID NO: 18), X=C(O)NH2, K=lysine, and H=histidine. Such polymers form a nanoparticle with the anti-TGF-beta 1 siRNA. The nanoparticle can be administered intravenously or intratumorally to the subject.

In another embodiment, the invention is directed to a method of killing cancer cells in a subject by administering to the subject a therapeutically effective amount of an immune checkpoint inhibitor with the therapeutically effective amount of the anti-TGF-beta 1 siRNA. In one aspect of this embodiment, the administration of the immune checkpoint inhibitor with the anti-TGF beta 1 siRNA increases the efficacy of the anti-TGF beta 1 siRNA.

In another embodiment, the invention is directed to a method of treating a cancer in a subject by administering to the subject a therapeutically effective amount of an immune checkpoint inhibitor with the therapeutically effective amount of the anti-TGF-beta 1 siRNA. In one aspect of this embodiment, the administration of the immune checkpoint inhibitor with the anti-TGF beta 1 siRNA increases the efficacy of the anti-TGF beta 1 siRNA.

As stated above, the immune checkpoint inhibitor and the anti-TGF-beta 1 siRNA are administered intravenously to the subject, into a tumor in the subject in proximity to the tumor, or systemically in a vehicle that allows delivery to the tumor.

In one aspect of this embodiment, the immune checkpoint inhibitor is a monoclonal antibody that blocks the interaction between receptors, such as PD-1, PD-L1, CTLA4, Lag3, and Tim3, and ligands for those receptors on mammalian cells, such as human cells. In a particular aspect, the monoclonal antibody is a monoclonal antibody to PD1 or PDL1. Examples of monoclonal antibodies include Atezoluzimab, Durvalumab, Nivolumab, Pembrolizumab, and Ipilimumab.

In still another aspect of this embodiment, the immune checkpoint inhibitor is a small molecule that blocks the interaction between receptors, such as PD-1, PD-L1, CTLA4, Lag3, and Tim3, and ligands for those receptors on mammalian cells, such as human cells. In a particular aspect, the small molecule blocks binding between PD1 and PDL1. BMS202 and similar ligands are examples of such small molecules.

In a further embodiment, the invention is directed to a method of killing cancer cells in a subject by administering to the subject a therapeutically effective amount of an anti-Cox-2 siRNA with the therapeutically effective amount anti-TGF-beta 1 siRNA. In one aspect of this embodiment, the combination is administered intravenously to the subject. In another aspect of this embodiment, the combination is administered into a tumor in a subject. In still another aspect of this embodiment, the combination is administered in proximity to the tumor or administered systemically in a vehicle that allows delivery to the tumor.

In a still further embodiment, the invention is directed to a method of treating a cancer in a subject by administering to the subject a therapeutically effective amount of an anti-Cox-2 siRNA with the therapeutically effective amount anti-TGF-beta 1 siRNA. In one aspect of this embodiment, the combination is administered intravenously to the subject. In another aspect of this embodiment, the combination is administered into a tumor in a subject. In still another aspect of this embodiment, the combination is administered in proximity to the tumor or administered systemically in a vehicle that allows delivery to the tumor.

The anti-Cox2 siRNA molecules include the sequences identified in Table 2.

TABLE 2
Anti-Cox2 siRNA Sequences

| | |
|---|---|
| hmCX-25-1: | sense 5′-r(GGUCUGGUGCCUGGUCUGAUGAUGU)-3′ (SEQ ID NO: 19) |
| | antisense 5′-r(ACAUCAUCAGACCAGGCACCAGACC)-3′ (SEQ ID NO: 20) |
| hmCX-25-2: | sense 5′-r(GAGCACCAUUCUCCUUGAAAGGACU)-3′ (SEQ ID NO: 21) |
| | antisense 5′-r(AGUCCUUUCAAGGAGAAUGGUGCUC)-3′ (SEQ ID NO: 22) |
| hmCX-25-3: | sense 5′-r(CCUCAAUUCAGUCUCUCAUCUGCAA)-3′ (SEQ ID NO: 23) |
| | antisense 5′-r(UUGCAGAUGAGAGACUGAAUUGAGG)-3′ (SEQ ID NO: 24) |
| hmCX25-4: | sense, 5′-r(GAUGUUUGCAUUCUUUGCCCAGCAC)-3′ (SEQ ID NO: 25) |
| | antisense, 5′-r(GUGCUGGGCAAAGAAUGCAAACAUC)-3′ (SEQ ID NO: 26) |
| hmCX25-5: | sense, 5′-r(GUCUUUGGUCUGGUGCCUGGUCUGA)-3′ (SEQ ID NO: 27) |
| | antisense, 5′-r(UCAGACCAGGCACCAGACCAAAGAC)-3′ (SEQ ID NO: 28) |
| hmCX25-6: | sense, 5′-r(GUGCCUGGUCUGAUGAUGUAUGCCA)-3′ (SEQ ID NO: 29) |
| | antisense, 5′-r(UGGCAUACAUCAUCAGACCAGGCAC)-3′ (SEQ ID NO: 30) |
| hmCX25-7: | sense, 5′-r(CACCAUUCUCCUUGAAAGGACUUAU)-3′ (SEQ ID NO: 31) |
| | antisense, 5′-r(AUAAGUCCUUUCAAGGAGAAUGGUG)-3′ (SEQ ID NO: 32) |
| hmCX25-8: | sense, 5′-r(CAAUUCAGUCUCUCAUCUGCAAUAA)-3′ (SEQ ID NO: 33) |
| | antisense, 5′-r(UUAUUGCAGAUGAGAGACUGAAUUG)-3′ (SEQ ID NO: 34) |

In one aspect, the anti-Cox2 siRNA comprises the following sequences: sense: 5′-ggucuggugccuggucugaugaugu-3′ (SEQ ID NO: 19); antisense: 5′-acaucaucagaccaggcaccagacc-3′ (SEQ ID NO: 20).

The anti-TGF-beta 1 siRNA and the anti-Cox2 siRNA are administered in a pharmaceutically acceptable carrier. Such carriers include branched histidine-lysine polymers. In one embodiment of such polymers, the polymer has the formula (R)K(R)-K(R)-(R)K(X), where R=KHHHKHHHKHHHKHHHK (SEQ ID NO: 17) or R=KHHHKHHHKHHHHKHHHK (SEQ ID NO: 18), X=C(O)NH2, K=lysine, H=histidine, and N=asparagine. Such polymers form a nanoparticle with the anti-TGF-beta 1 siRNA and the anti-Cox2 siRNA. The nanoparticle can be administered intravenously or intratumorally to the subject.

As stated above with respect to the anti-TGF-beta 1 siRNA, the cancer (and the cancer cells) targeted by the combination of anti-TGF-beta 1 siRNA and anti-Cox2 siRNA can be any cancer that afflicts a subject. Such cancers include liver, colon, pancreatic, lung, and bladder cancer. The liver cancer can be a primary liver cancer or a cancer that has metastasized to the liver from another tissue. Primary liver cancers include hepatocellular carcinoma and hepatoblastoma. Metastasized cancers include colon and pancreatic cancer.

In a further embodiment, the invention is directed to a method of killing cancer cells in a subject by administering to the subject a therapeutically effective amount of an immune checkpoint inhibitor with a therapeutically effective amount of an anti-TGF-beta 1 siRNA and a therapeutically effective amount of an anti-Cox2 siRNA. The cancer cells and cancers to which this method is directed are any cancer that afflicts a subject, including those described above. In one aspect of this embodiment, the administration of an immune checkpoint inhibitor with the anti-TGF-beta 1 siRNA and the anti-Cox2 siRNA increases the efficacy of either one of the siRNA's alone.

In a still further embodiment, the invention is directed to a method of treating a cancer in a subject by administering to the subject a therapeutically effective amount of an immune checkpoint inhibitor with a therapeutically effective amount of an anti-TGF-beta 1 siRNA and a therapeutically effective amount of an anti-Cox2 siRNA. The cancer cells and cancers to which this method is directed are any cancer that afflicts a subject, including those described above. In one aspect of this embodiment, the administration of an immune checkpoint inhibitor with the anti-TGF-beta 1 siRNA and the anti-Cox2 siRNA increases the efficacy of either one of the siRNA's alone.

As stated above, the immune checkpoint inhibitor, the anti-TGF-beta 1 siRNA, and the anti-Cox2 siRNA are administered intravenously to the subject, into a tumor in the subject in proximity to the tumor, or systemically in a vehicle that allows delivery to the tumor.

The immune checkpoint inhibitor administered with the combination of the siRNA molecules is a monoclonal antibody or a small molecule as described above. It can be administered before, after, or concurrently with the combination of the siRNA molecules.

The invention is directed to certain pharmaceutical compositions. In one embodiment, the composition comprises an anti-TGF-beta 1 siRNA as described herein in a pharmaceutically acceptable carrier as described herein.

In another embodiment, this pharmaceutical composition is used in connection with an immune checkpoint inhibitor as described herein. Thus, this embodiment of the invention is directed to a combination of therapeutic drugs comprising an immune checkpoint inhibitor and a pharmaceutical composition comprising an anti-TGF-beta 1 siRNA in a pharmaceutically acceptable carrier as described herein.

In still another embodiment, the invention is directed to a combination of therapeutic drugs comprising an immune checkpoint inhibitor and a pharmaceutical composition comprising an anti-TGF-beta 1 siRNA and an anti-Cox2 siRNA and a pharmaceutically acceptable carrier as described herein.

The therapeutic drug combination described herein is also useful for enhancing the anti-tumor efficacy of an immune checkpoint inhibitor in a subject with a cancer. A therapeutically effective amount of a pharmaceutical composition comprising an anti-TGF-beta 1 siRNA and an anti-Cox2 siRNA is administered to the subject along with a therapeutically effective amount of a checkpoint inhibitor. The anti-TGF-beta 1 siRNA decreases the subject's inflammatory response to the cancer and allows better penetration of T-cells and other immune cells into the tumor. It also creates a stronger immune response to the cancer in the subject than the immune response created by the checkpoint inhibitor alone. This response involves greater T-cell activation and penetration into the cancer. The anti-Cox2 siRNA decreases the subject's inflammatory response to the cancer and/or decreases the formation of exhausted T-cells or regulatory T-cells around the cancer.

The therapeutic drug combination described herein is also useful for antigenically priming T cells to recognize and kill cancer cells in a subject and for promoting T-cell-mediated immunity against a cancer in a subject. A therapeutically effective amount of the combination is administered to the subject. The cancers are those described herein.

In one particular embodiment, the invention is directed to a method of treating a liver cancer in a subject by administering to the subject a therapeutically effective amount of a pharmaceutical composition of the invention or a therapeutic drug combination of the invention. In one aspect of this embodiment, the liver cancer is a primary liver cancer. In a particular aspect, the primary liver cancer is a hepatocellular carcinoma or a hepatoblastoma. In another aspect of this embodiment, the liver cancer is a cancer that has metastasized to the liver from another tissue in the subject's body. Such metastasized cancers include colon cancer and pancreatic cancer. In one aspect of this embodiment, the subject is a human.

In another particular embodiment, the invention is directed to a method of killing hepatocellular carcinoma cells in a human comprising administering to the human a therapeutically effective amount of a pharmaceutical composition comprising an anti-TGF-beta 1 siRNA and an anti-Cox2 siRNA in a pharmaceutically acceptable carrier comprising a branched histidine-lysine polymer that forms a nanoparticle with the anti-TGF-beta 1 siRNA and the anti-Cox2 siRNA, wherein the anti-TGF-beta 1 siRNA comprises the sequences: sense: 5′-cccaagggcuaccaugccaacuucu-3′ (SEQ ID NO: 3); antisense: 5′-agaaguuggcaugguagcccuuggg-3′ (SEQ ID NO: 4) and the anti-Cox2 siRNA comprises the sequences: sense: 5′-ggucuggugccuggucugaugaugu-3′ (SEQ ID NO: 19); antisense: 5′-acaucaucagaccaggcaccagacc-3′ (SEQ ID NO: 20).

In still another particular embodiment, the invention is directed to a method of killing hepatocellular carcinoma cells in a human comprising administering to the human a therapeutically effective amount of an immune checkpoint inhibitor and a pharmaceutical composition comprising an anti-TGFbeta 1 siRNA and an anti-Cox2 siRNA in a pharmaceutically acceptable carrier comprising a branched histidine-lysine polymer that forms a nanoparticle with the anti-TGF-beta 1 siRNA and the anti-Cox2 siRNA, wherein the anti-TGF-beta 1 siRNA comprises the sequences: sense: 5′-cccaagggcuaccaugccaacuucu-3′ (SEQ ID NO: 3); antisense: 5′-agaaguuggcaugguagcccuuggg-3′ (SEQ ID NO: 4) and the anti-Cox2 siRNA comprises the sequences: sense: 5′-ggucuggugccuggucugaugaugu-3′ (SEQ ID NO: 19); antisense: 5′-acaucaucagaccaggcaccagacc-3′ (SEQ ID NO: 20) and wherein the checkpoint inhibitor comprises a monoclonal antibody able to bind to and block interactions between PD1 and PDL1. Such monoclonal antibodies include Atezoluzimab, Durvalumab, Nivolumab, Pembrolizumab, and Ipilimumab.

In a further particular embodiment, the invention is directed to a combination of therapeutic drugs comprising an immune checkpoint inhibitor and a pharmaceutical composition comprising an anti-TGF-beta 1 siRNA and an anti-Cox 2 siRNA in a pharmaceutically acceptable carrier, wherein the checkpoint inhibitor comprises a monoclonal antibody selected from the group consisting of Atezoluzimab, Durvalumab, Nivolumab, Pembrolizumab, and Ipilimumab, the anti-TGF-beta 1 siRNA comprises the sequences: sense: 5′-cccaagggcuaccaugccaacuucu-3′ (SEQ ID NO: 3); antisense: 5′-agaaguuggcaugguagcccuuggg-3′ (SEQ ID NO: 4), the anti-Cox2 siRNA comprises the sequences: sense: 5′-ggucuggugccuggucugaugaugu-3′ (SEQ ID NO: 19); antisense: 5′-acaucaucagaccaggcaccagacc-3′ (SEQ ID NO: 20), and the pharmaceutically acceptable carrier comprises a branched histidine-lysine polymer that forms a nanoparticle with the anti-TGF-beta 1 siRNA and the anti-Cox2 siRNA. In one aspect of this embodiment, the branched histidine-lysine polymer has the formula (R)K(R)-K(R)-(R)K(X), where R=KHHHKHHHKHHHKHHHK (SEQ ID NO: 17) or KHHHKHHHKHHHHKHHHK (SEQ ID NO: 18), X=C(O)NH2, K=lysine, H=histidine, and N=asparagine.

### Definitions

Liver cancer is any primary cancer within the liver, i.e., one that starts in the liver; or any secondary cancer within the liver, i.e., a cancer that metastasizes to the liver from another tissue in the mammal's body. An example of a primary liver cancer is hepatocellular carcinoma. An example of a secondary liver cancer is a colon cancer.

A cancer is any malignant tumor.

A malignant tumor is a mass of neoplastic cells.

Treating/treatment is killing some or all of the cancer cells, reducing the size of the cancer, inhibiting the growth of the cancer, or reducing the growth rate of the cancer in a subject.

Anti-TGF-beta 1 siRNA is an siRNA molecule that reduces or prevents the expression of the gene in a mammalian cell that codes for the synthesis of TGF-beta 1 protein.

Anti-Cox2 siRNA is an siRNA molecule that reduces or prevents the expression of the gene in a mammalian cell that codes for the synthesis of Cox2 protein.

An siRNA molecule is a duplex oligonucleotide, that is a short, double-stranded polynucleotide, that interferes with the expression of a gene in a cell, after the molecule is introduced into the cell. For example, it targets and binds to a complementary nucleotide sequence in a single stranded target RNA molecule. SiRNA molecules are chemically synthesized or otherwise constructed by techniques known to those skilled in the art. Such techniques are described in U.S. Pat. Nos. 5,898,031, 6,107,094, 6,506,559, 7,056,704 and in European Pat. Nos. 1214945 and 1230375, which are incorporated herein by reference in their entireties. By convention in the field, when an siRNA molecule is identified by a particular nucleotide sequence, the sequence refers to the sense strand of the duplex molecule. One or more of the ribonucleotides comprising the molecule can be chemically modified by techniques known in the art. In addition to being modified at the level of one or more of its individual nucleotides, the backbone of the oligonucleotide can be modified. Additional modifications include the use of small molecules (e.g. sugar molecules), amino acids, peptides, cholesterol, and other large molecules for conjugation onto the siRNA molecule.

A branched histidine-lysine polymer is a peptide consisting of histidine and lysine amino acids. By synthesizing multiple amino acids from a common lysine core, the peptide is composed of 4 arms or branches. Such polymers are described in U.S. Pat. No. 7,070,807 B2, issued Jul. 4, 2006, U.S. Pat. No. 7,163,695 B2, issued Jan. 6, 2007, and U.S. Pat. No. 7,772,201 B2, issued Aug. 10, 2010, which are incorporated herein by reference in their entireties.

An immune checkpoint inhibitor is a drug that blocks certain proteins made by some types of immune system cells, such as T cells, and some cancer cells. These checkpoint proteins help keep immune responses in check and can keep T cells from killing the cancer cells. When these checkpoint proteins are blocked, the “brakes” on the immune system are released, and T cells are able to kill cancer cells better. Examples of checkpoint proteins found on T cells or cancer cells include PD-1/PD-L1 and CTLA-4/B7-1/B7-2.

In proximity to the cancer means in tissue or cells close to or surrounding a tumor or series of tumor cells.

Enhancing the antitumor efficacy means providing a greater reduction in growth rate of the tumor cells, greater effect in killing the tumor cells and/or reducing tumor mass and eventually producing a better therapeutic effect by prolonging life of the subject with the tumor. Such effects may be mediated by a direct action on the tumor cells themselves or an augmentation of the activity of the T-cells or a mechanism by which the T-cells are afforded better access to the tumor cells and/or are activated to promote a stronger immune reaction against the tumor with or without an increase in the ability to recognize tumor cells even after the initial treatment.

The following examples illustrate certain aspects of the invention and should not be construed as limiting the scope thereof.

### EXAMPLES

### Experimental Results

STP707 consists of 2 siRNAs (targeting TGF-beta 1 and Cox2 genes) protected by a polypeptide delivery nanoparticle consisting of the branched polypeptide HKP (Histidine Lysine Polymer). STP707 is described in U.S. Pat. No. 9,642,873 B2, dated May 9, 2017, and US Reissued Patent RE46,873 E, dated May 29, 2018, the disclosures of which are incorporated by reference herein in their entireties.

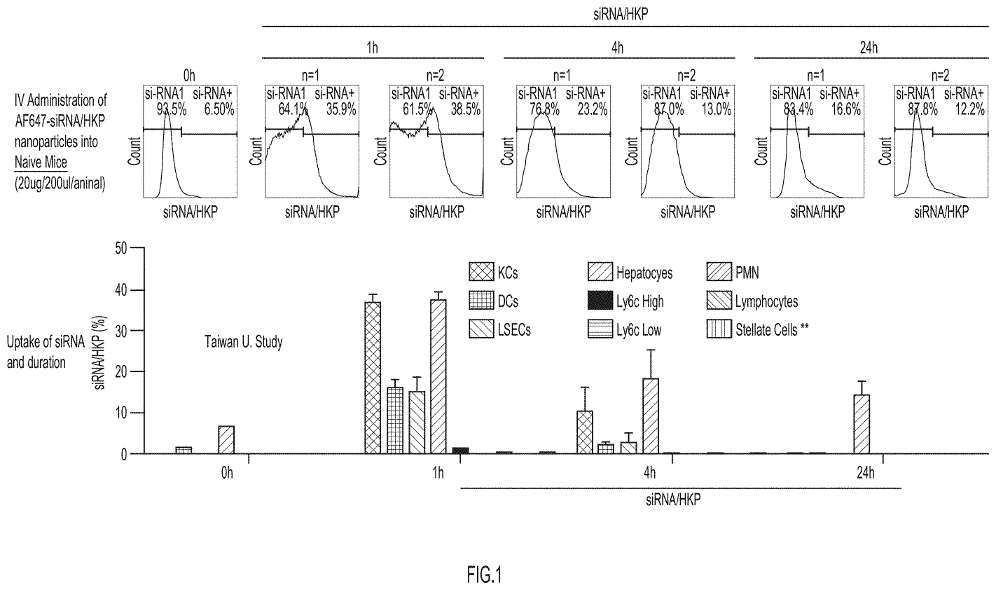

Using a branched polypeptide nanoparticle consisting of histidine and lysine amino acids (HKP), we have demonstrated that IV injection allows the nanoparticles to be taken up with higher efficiency by cells within the liver. Using flow cytometry to measure the uptake of fluorescent tagged siRNAs from the nanoparticles, we have demonstrated delivery to specific cell types within the liver, including the Stellate cells, LSEC cells, and the hepatocytes as well as the Kupffer cells (FIG. 1). This would therefore suggest that we can get very good delivery efficiency of siRNAs to these cells within the liver and can therefore silence targets of interest within these cells.

We examined STP707, as a monotherapy and in combination with an anti-PD-L1 monoclonal antibody (mAb) (anti-PD-L1 monoclonal Ab Clone 10F.9G2 from BioXcell, West Lebanon, N.H. 03784), in an orthotopic murine hepatocellular carcinoma model using a bioluminescent variant of the Hepa 1-6 cell line to allow monitoring for tumor load over time.

We used an orthotopic implant model, where HCC liver cancer tumor cells (Hepa 1-6 cells) were surgically implanted into the organ from which they were derived (the liver). The mouse liver cancer cell line was modified to express luciferase (Hepa 1-6-Lux). Then, upon addition of the substrate to the animals, the degree of growth of the tumors in the livers of these animals could be monitored using a luminescence detection system. This allows measurement of the tumor growth in a non-invasive manner that is not harmful to the animals. We monitored the rate of growth of the tumors using this method. We examined the rate of growth in the absence of any treatment (Control group), using the gold standard of care for hepatocellular carcinoma treatment in humans (Sorafenib—a kinase inhibitor; 50 mgs/Kg administered QD) and a validated mouse anti-PDL1 antibody shown to inhibit tumor growth in animals with orthotopic Hepa1-6 tumors (administered BIW at 5 mgs/Kg).

We also compared the effect of siRNAs shown to inhibit TGF-beta and Cox2 (STP707) delivered using the HKP peptide nanoparticles (administered IV BIW at a dose of 40 ug or 20 ug per injection). We further analyzed the effect of STP707 when administered along with the anti-PDL1 antibody.

We examined STP707 for efficacy in treating HCC using a syngeneic, orthotopic murine hepatocellular carcinoma model using a bioluminescent variant of the Hepa 1-6 cell line to allow monitoring for tumor load over time.

The experiments were performed using the C57BL/6J mouse strain from Charles River labs.

Vehicle (HKP+Non-silencing siRNA; control) or STP707 were both administered intravenously (iv).

8 animals were randomly assigned to each treatment group. The treatment groups were as follows:
1. Vehicle only
2. Sorafenib (50 mg/Kg) p.o., QD
3. Anti-PD-L1 (5 mgs/Kg), i.p., BIW
4. Anti-PD-L1 (5 mgs/Kg), i.p., BIW+20 ugs STP707/injection iv BIW
5. Anti-PD-L1 (5 mgs/Kg), i.p., BIW+40 ugs STP707/injection iv BIW
6. 40 ugs STP707/injection iv BIW alone

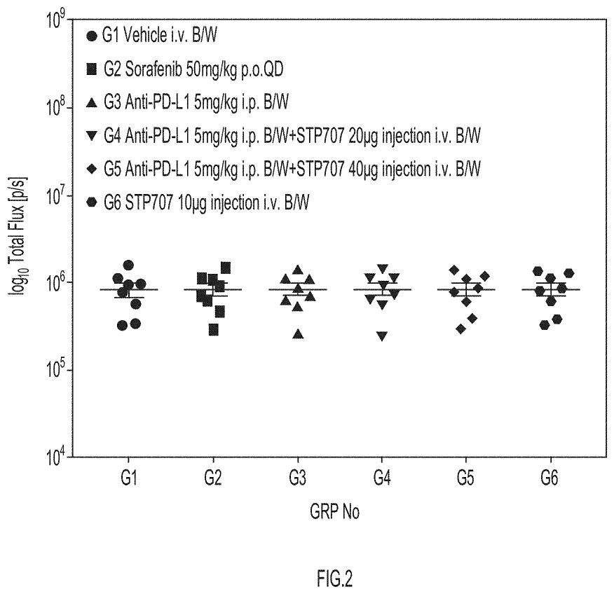

The animals were randomized at the outset of the experiment based on weight. The randomization provided very similar weight distributions in the selected animals within each group as shown in FIG. 2.

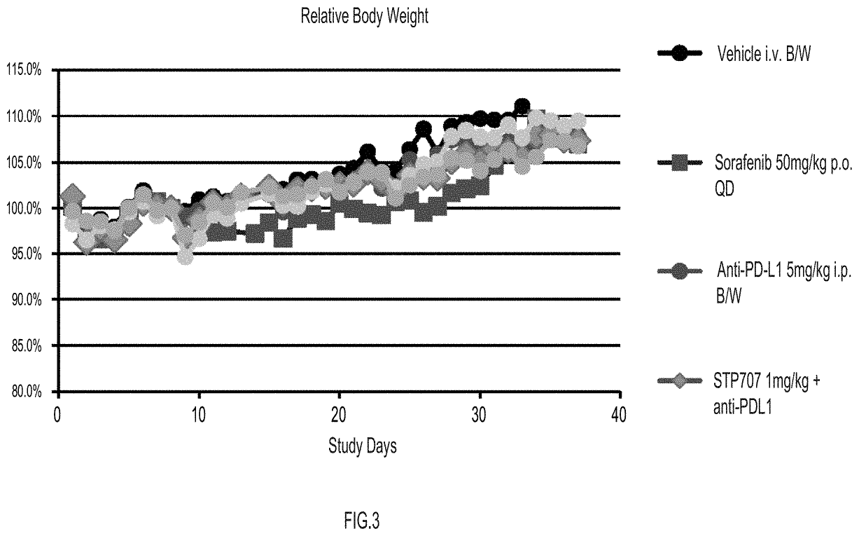

The bodyweights of the animals were measured daily during the dosing phase of the efficacy study. Mean body weights of each group were plotted (FIG. 3). Sorafenib alone induced a slight change in body weights. However, all other treatment schemes (STP707 alone or +anti-PDL1) were well tolerated and no significant body weight loss was observed in the treatment arms.

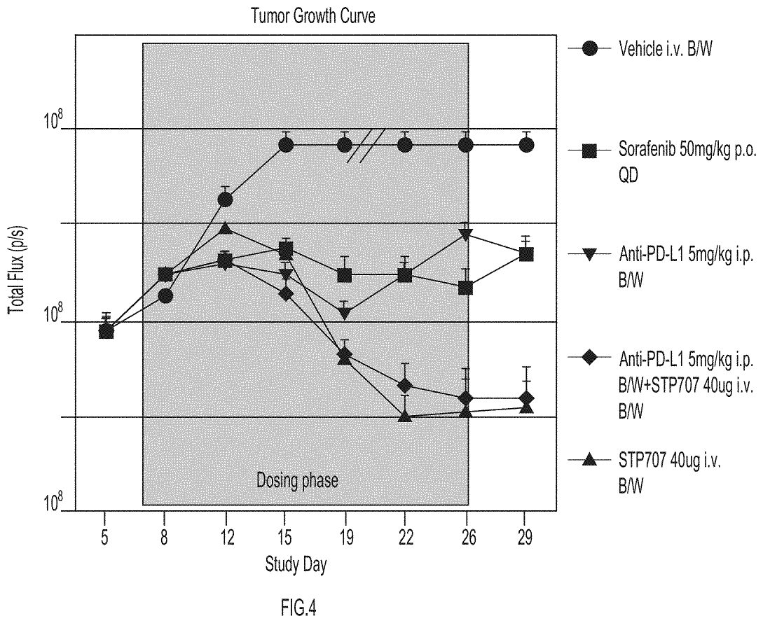

Tumor growth was monitored by bioluminescence imaging and reflected by tumor associated bioluminescence (TABL) quantification. TABL was plotted by study day and shown in FIG. 4.

Upon completion of the scheduled dosing phase, tumor outgrowth was monitored for groups 2-6. No tumor regrowth was observed prior to the last day of the study (day 50), suggesting that the treatments were very efficacious at inhibiting tumor growth and preventing regrowth suggesting a pronounced effect on tumor viability.

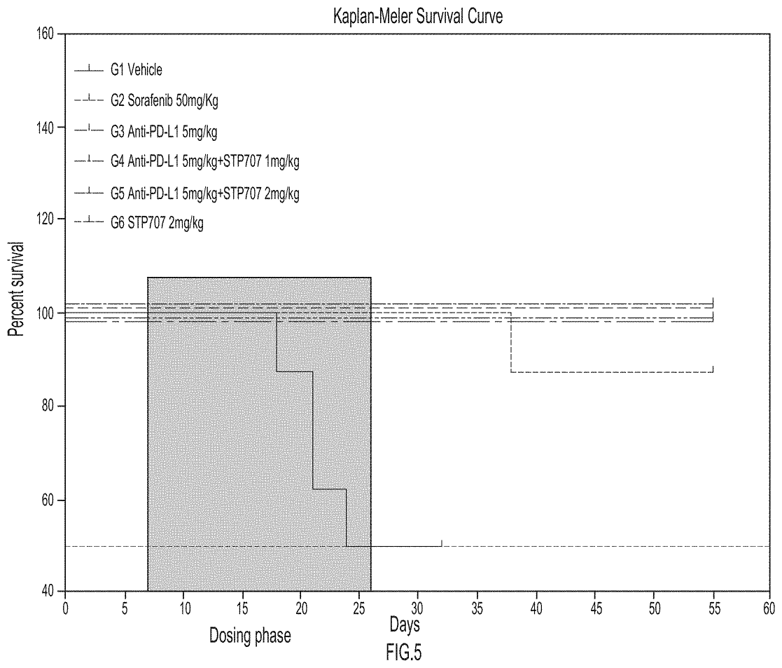

The effect of the combination therapies was further emphasized by survival analysis (using humane surrogate endpoints) on all mice in the study (FIG. 5). The endpoint for humane termination was defined as either tumors reaching a maximum permissible size or when tumors displayed adverse clinical signs. All treatment regimens produced statistically significantly improved survival as demonstrated by Log-rank (Mantel-Cox) Test (p=0.0001) and Gehan-Breslow-Wilcoxon Test (p=0.0002). No differences in survival were observed among the treatment groups.

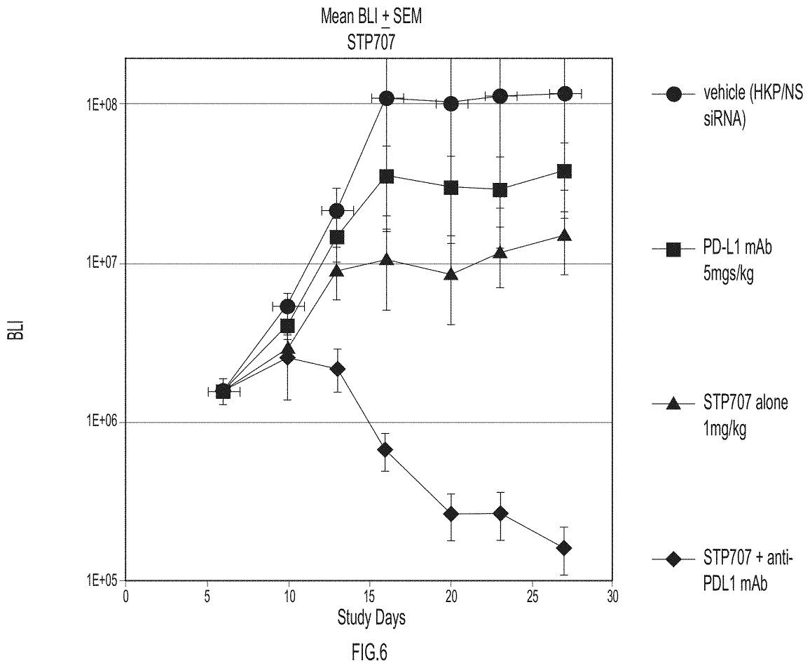

To validate the results obtained above, we repeated the study using a lower dose of STP707 (1 mg/kg) (FIG. 6).

The data obtained supported the observation that STP707 shows single agent action against the tumor—diminishing growth relative to the control (untreated) cohort. The STP707 arm showed better efficacy than the antibody arm (PDL1) alone.

In this study, STP707 shows single agent activity better than anti-PDL1 antibody alone. However, combining STP707 with the antibody treatment reduced the tumor to undetectable levels after 4 doses.

Since there was no tumor evident in either of the treatment groups with STP707, we repeated the study but using only 3 doses of STP707 at 1 mg/Kg. After the third dose, animals were euthanized, livers removed and sectioned and stained for CD4+ and CD8+ T-cells using Immunohistochemistry.

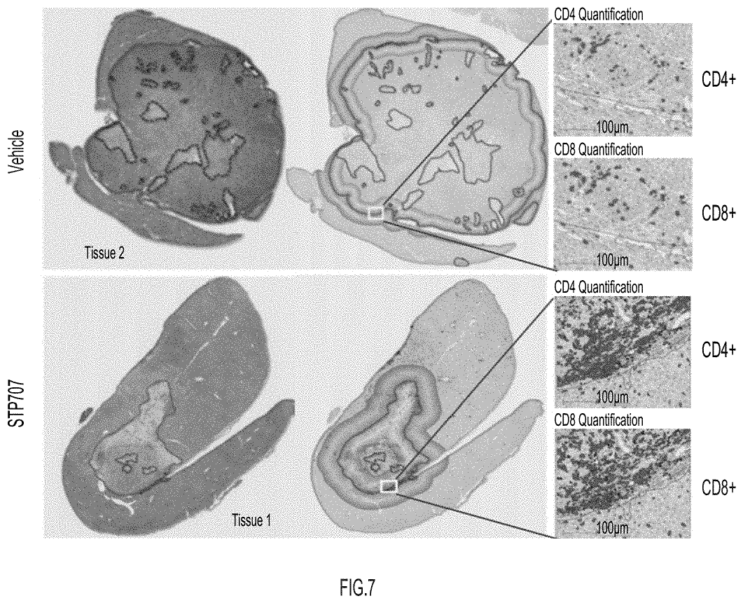

Even in these reduced dose studies, we see a dramatic difference between untreated (control) samples and STP707 treated samples in terms of the overall tumor size. In untreated animals the tumor is almost completely the size of the liver, while in STP707 samples the tumor was much reduced (FIG. 7).

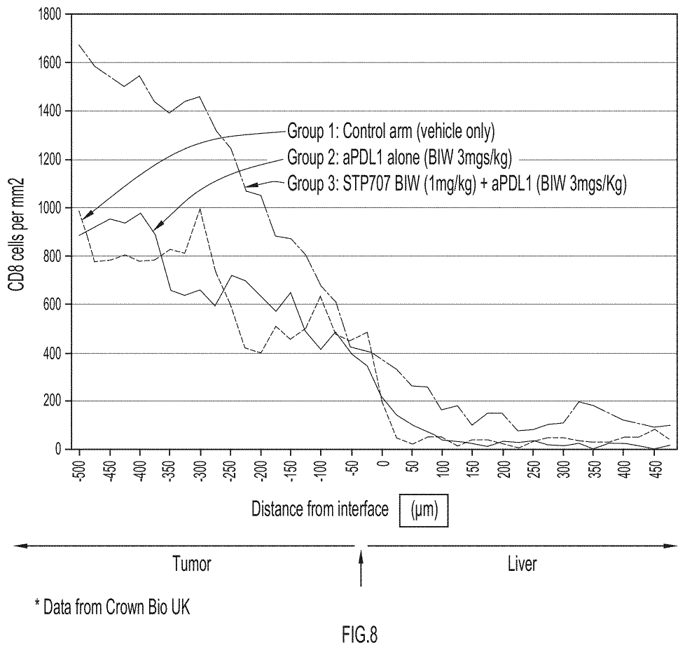

Furthermore, IHC staining for CD4+ and CD8+ T-cells showed a dramatic increase in these T-cells penetrating the tumor of the STP707 treated sample (FIG. 7 and FIG. 8). Image analysis was performed to quantitate the CD4+ and CD8+ T-cells at the margins between the tumor and the liver as shown by the colored lines in the tumor samples. These lines are drawn at 50 um distances away from the tumor margin—either in towards the tumor or out, away from the tumor but towards the liver. Image analysis counted all CD8+ T-cells in each 50 um segment and the data was plotted as shown in FIG. 8.

STP707 treatment together with the anti-PDL1 Ab showed a 2-fold increase in CD8+ T-cells at 500 um in to the tumor. It also shows an increase in CD8+ T-cells within the liver close to the tumor—suggesting possible recruitment of Tcells induced by the lowering of the TGF-beta “wall” surrounding the tumor.

### CONCLUSIONS

The primary objective of this study was to determine the tolerability and efficacy of STP707 (HKP polypeptide nanoparticles containing siRNAs against TGF beta1 and Cox2), as a monotherapy and in combination with anti-PD-L1, in an orthotopic murine hepatocellular carcinoma model using a bioluminescent variant of the Hepa 1-6 cell line.

All treatment regimens were well tolerated at the doses and formulations tested; no adverse clinical signs or body weight loss were noted in any of the treatment groups. All treatment regimens produced statistically significant reduced tumor growth when compared with control. However, STP707 monotherapy (2 mgs/kg) resulted in reduced tumor growth that was also observed when combined with the anti-PDL1 Ab. Reducing the dose of STP707 to 1 mg/Kg demonstrated a lower effect of the STP707 alone but now the additivity with the anti-PDL1 Ab was more evident.

Anti-PD-L1 5 mg/kg and STP705 (20 ug per injection (1 mg/kg)) combination appeared to be more efficacious than STP705 monotherapy which is more efficacious than Anti-PD-L1 5 mg/kg.

Our results show that STP707 augments the action of the anti-PDL1 antibody—resulting in a dramatic reduction of tumor viability with no return of the tumor cells—even after stopping dosing for 2+ weeks. The fact that we show delivery to the liver with this formulation given IV suggests we can treat tumors that are harbored in the liver with this regimen. This would include any tumors that naturally occur in the liver (hepatoblastoma or Hepatocellular carcinoma (HCC)) or tumors that metastasize to the liver (e.g. colon cancer).

Furthermore, in previous studies, we have demonstrated the ability to reduce gene expression for these 2 gene targets when the product is administered by injection (e.g. intradermally). This would suggest that we will be able to get the same therapeutic benefit of promoting an immune response to the tumors when the product is administered via injection in proximity to the tumor. This could be used for dermal cancers (e.g. nonmelanoma skin cancers or melanoma tumors in the skin), or in tumors in other organs where material can be injected close to the tumor site to promote the same effect.

### REFERENCES

[1] Liu et al., Cancer Cell Int (2015) 15:106-112 “Cyclooxygenase-2 promotes tumor growth and suppresses tumor immunity”
[2] Zelenay et al., Cell (2015) 162: 1257-1270 “Cyclooxygenase-Dependent Tumor Growth through Evasion of Immunity”
[3] Mariathasan et al., Nature (2018) 554; 544-548 “TGFβ attenuates tumour response to PD-L1 blockade by contributing to exclusion of T cells”

All publications identified herein, including issued patents and published patent applications, and all database entries identified by url addresses or accession numbers are incorporated herein by reference in their entireties.

Although this invention has been described in relation to certain embodiments thereof, and many details have been set forth for purposes of illustration, it will be apparent to those skilled in the art that the invention is susceptible to additional embodiments and that certain of the details described herein may be varied without departing from the basic principles of the invention.

1. A method of killing cancer cells in a subject comprising administering to the subject a therapeutically effective amount of an anti-TGF-beta 1 siRNA wherein said anti-TGF-beta 1 siRNA is administered intravenously or intratumorally or in proximity to the tumor.

2-3. (canceled)

4. The method of claim 1, wherein the cancer is selected from the group consisting of liver, colon, pancreatic, lung, or bladder cancer.

5-7. (canceled)

8. The method of claim 4, wherein the cancer is liver cancer, primary liver cancer, Hepatocellular cancer or a hepatoblastoma, or has metastasized to the liver from another tissue in the subject's body.

9. The method of claim 8, wherein the metastasized cancer comprises a colon cancer, a pancreatic cancer or a lung cancer.

10. (canceled)

11. The method of claim 1, wherein the anti-TGF-beta 1 siRNA is selected from the sequences in Table 1.

12. The method of claim 1, wherein the anti TGF beta 1 siRNA comprises the sequences: sense: 5′-cccaagggcuaccaugccaacuucu-3′ (SEQ ID NO: 3); antisense: 5′-agaaguuggcaugguagcccuuggg-3′ (SEQ ID NO: 4) and is administered in a pharmaceutically acceptable carrier comprising a branched histidine-lysine polymer.

13-14. (canceled)

15. The method of claim 14, wherein the branched histidine-lysine polymer has the formula (R)K(R)-K(R)-(R)K(X), where R=KHHHKHHHKHHHKHHHK (SEQ ID NO: 17) or R=KHHHKHHHKHHHHKHHHK (SEQ ID NO: 18), X=C(O)NH2, K=lysine, H=histidine and forms a nanoparticle with the anti-TGF-beta 1 siRNA.

16. (canceled)

17. The method of claim 15 wherein the nanoparticle is administered intravenously to the subject.

18. The method of claim 1, comprising administering a therapeutically effective amount of an anti-Cox2 siRNA with the therapeutically effective amount of the anti-TGF-beta 1 siRNA.

19. The method of claim 18, wherein the anti-Cox2 siRNA is selected from the sequences in Table 2.

20. The method of claim 18, wherein the anti-Cox2 siRNA comprises the sequences:
sense:
(SEQ ID NO: 19)
5′-ggucuggugccuggucugaugaugu-3′;
antisense:
(SEQ ID NO: 20)
5′-acaucaucagaccaggcaccagacc-3′.

21. The method of claim 1, wherein the anti-TGF-beta 1 siRNA and the anti-Cox2 siRNA are administered in a pharmaceutically acceptable carrier, wherein the pharmaceutically acceptable carrier comprises a branched histidine-lysine polymer and the branched histidine-lysine polymer has the formula (R)K(R)-K(R)-(R)K(X), where R=KHHHKHHHKHHHKHHHK (SEQ ID NO: 17) or R=KHHHKHHHKHHHHKHHHK (SEQ ID NO: 18), X=C(O)NH₂, K=lysine, H=histidine and forms a nanoparticle with the anti-TGF-beta 1 siRNA and the anti-Cox2 siRNA.

22-24. (canceled)

25. The method of claim 23, wherein the nanoparticle is administered intravenously to the subject.

26. The method of claim 1, further comprising administering a therapeutically effective amount of an immune checkpoint inhibitor with the therapeutically effective amount of the anti-TGF-beta 1 siRNA.

27-30. (canceled)

30. The method of claim 26, wherein the immune checkpoint inhibitor comprises a monoclonal antibody and wherein the monoclonal antibody blocks the interaction between receptors on a subject cell selected from the group consisting of PD-1, PD-L1, CTLA4, Lag3, and Tim3 and the ligands for those receptors.

31-32. (canceled)

33. The method of claim 30, wherein the monoclonal antibody is selected from the group consisting of Atezoluzimab, Durvalumab, Nivolumab, Pembrolizumab, and Ipilimumab.

34. The method of claim 26, wherein the immune checkpoint inhibitor comprises a small molecule that blocks the interaction between receptors on a subject cell selected from the group consisting of PD-1, PD-L1, CTLA4, Lag3, and Tim3 and the ligands for those receptors.

35-36. (canceled)

37. The method of claim 34, wherein the small molecule is selected from the group consisting of BMS202 and similar ligands.

38. A combination of therapeutic drugs comprising an immune checkpoint inhibitor and a pharmaceutical composition comprising an anti-TGF-beta 1 siRNA in a pharmaceutically acceptable carrier, wherein said anti-TGF-beta 1 siRNA is selected from the sequences in Table 1.

39. (canceled)

40. The combination of claim 38, wherein the anti-TGF-beta 1 siRNA comprises the sequences: sense: 5′-cccaagggcuaccaugccaacuucu-3′ (SEQ ID NO: 3); antisense: 5′-agaaguuggcaugguagcccuuggg-3′ (SEQ ID NO: 4).

41. The combination of claim 38, wherein the pharmaceutically acceptable carrier comprises a branched histidine-lysine polymer forms a nanoparticle with the anti-TGF-beta 1 siRNA, wherein said branched histidine-lysine polymer has the formula (R)K(R)-K(R)-(R)K(X), where R=KHHHKHHHKHHHKHHHK (SEQ ID NO: 17) or R=KHHHKHHHKHHHHKHHHK (SEQ ID NO: 18), X=C(O)NH2.

42. (canceled)

43. A combination of therapeutic drugs comprising an immune checkpoint inhibitor and a pharmaceutical composition comprising an anti-TGF-beta 1 siRNA and an anti-Cox2 siRNA in a pharmaceutically acceptable carrier, wherein the anti-TGF-beta 1 siRNA comprises the sequences: sense: 5′-cccaagggcuaccaugccaacuucu-3′ (SEQ ID NO: 3); antisense: 5′-agaaguuggcaugguagcccuuggg-3′ (SEQ ID NO: 4) and the anti-Cox2 siRNA comprises the sequences: sense: 5′-ggucuggugccuggucugaugaugu-3′ (SEQ ID NO: 19); antisense: 5′-acaucaucagaccaggcaccagacc-3′ (SEQ ID NO: 20), and wherein the pharmaceutically acceptable carrier comprises a branched histidine-lysine polymer with the formula (R)K(R)-K(R)-(R)K(X), where R=KHHHKHHHKHHHKHHHK (SEQ ID NO: 17) or KHHHKHHHKHHHHKHHHK (SEQ ID NO: 18), X=C(O)NH₂, and wherein said carrier forms a nanoparticle with the anti-TGF-beta 1 siRNA and the anti-Cox2 siRNA.

44-47. (canceled)

48. The combination of claim 38, wherein the immune checkpoint inhibitor comprises a monoclonal antibody that blocks the interaction between receptors on a subject cell selected from the group consisting of PD-1, PD-L1, CTLA4, Lag3, and Tim3 and the ligands for those receptors.

49-50. (canceled)

51. The combination of claim 48, wherein the monoclonal antibody is selected from the group consisting of Atezoluzimab, Durvalumab, Nivolumab, Pembrolizumab, and Ipilimumab.

52. The combination of claim 38, wherein the immune checkpoint inhibitor comprises a small molecule that blocks the interaction between receptors on a subject cell selected from the group consisting of PD-1, PD-L1, CTLA4, Lag3, and Tim3 and the ligands for those receptors.

53-55. (canceled)

56. A method of enhancing the anti-tumor efficacy of an immune checkpoint inhibitor in a subject with a cancer comprising administering to the human a therapeutically effective amount of an anti-TGF-beta 1 siRNA and an anti-Cox2 siRNA with a checkpoint inhibitor, wherein the anti-TGF-beta 1 siRNA decreases the subject's inflammatory response to the cancer and allows better penetration of T-cells and other immune cells into the tumor.

57-63. (canceled)

64. A method for promoting T-cell-mediated immunity against a cancer in a subject, comprising administering to the subject a therapeutically effective amount of the combination of claim 38.

65-77. (canceled)

78. A method of killing hepatocellular carcinoma cells in a human comprising administering to the human a therapeutically effective amount of a pharmaceutical composition comprising an anti-TGF-beta 1 siRNA and an anti-Cox2 siRNA in a pharmaceutically acceptable carrier comprising a branched histidine-lysine polymer that forms a nanoparticle with the anti-TGF-beta 1 siRNA and the anti-Cox2 siRNA, wherein the anti-TGF-beta 1 siRNA comprises the sequences: sense: 5′-cccaagggcuaccaugccaacuucu-3′ (SEQ ID NO: 3); antisense: 5′-agaaguuggcaugguagcccuuggg-3′ (SEQ ID NO: 4) and the anti-Cox2 siRNA comprises the sequences: sense: 5′-ggucuggugccuggucugaugaugu-3′ (SEQ ID NO: 19); antisense: 5′-acaucaucagaccaggcaccagacc-3′ (SEQ ID NO: 20), and wherein the checkpoint inhibitor comprises a monoclonal antibody able to bind to and block interactions between PD1 and PDL1 selected from the group consisting of Atezoluzimab, Durvalumab, Nivolumab, Pembrolizumab, and Ipilimumab.

79. (canceled)

80. A combination of therapeutic drugs comprising an immune checkpoint inhibitor and a pharmaceutical composition comprising an anti-TGF-beta 1 siRNA and an anti-Cox2 siRNA in a pharmaceutically acceptable carrier, wherein the checkpoint inhibitor comprises a monoclonal antibody selected from the group consisting of Atezoluzimab, Durvalumab, Nivolumab, Pembrolizumab, and Ipilimumab, the anti-TGF-beta 1 siRNA comprises the sequences: sense: 5′-cccaagggcuaccaugccaacuucu-3′ (SEQ ID NO: 3); antisense: 5′-agaaguuggcaugguagcccuuggg-3′ (SEQ ID NO: 4), the anti-Cox2 siRNA comprises the sequences: sense: 5′-ggucuggugccuggucugaugaugu-3′ (SEQ ID NO: 19); antisense: 5′-acaucaucagaccaggcaccagacc-3′ (SEQ ID NO: 20), and the pharmaceutically acceptable carrier comprises a branched histidine-lysine polymer that forms a nanoparticle with the anti-TGF-beta 1 siRNA and the anti-Cox2 siRNA.

81. (canceled)
